(19) United States
(12) Patent Application Publication
(10) Pub. No.: US 2006/0116666 A1
(43) Pub. Date: Jun. 1, 2006

(54) Two-stage scar generation for treating atrial fibrillation

(75) Inventors: Richard Cornelius, Wayzata, MN (US); William Swanson, St. Paul, MN (US); Daniel Sullivan, Plymouth, MN (US); Ronald Shebuski, Bergland, MI (US)

Correspondence Address: INSKEEP INTELLECTUAL PROPERTY GROUP, INC, 2281 W. 190TH STREET, SUITE 200, TORRANCE, CA, 90504, US

(73) Assignee: Sinus Rhythm Technologies, Inc.

(21) Appl. No.: 11/246,412

(22) Filed: Oct. 7, 2005

Related U.S. Application Data
(60) Provisional application No. 60/617,260, filed on Oct. 8, 2004.
(60) Provisional application No. 60/664,925, filed on Mar. 24, 2005.

(51) Int. Cl. A61B 17/00; A61B 18/18

(52) U.S. Cl. 606/1; 606/41

(57) ABSTRACT

The present invention seeks to provide an implant configured to utilize at least two different scar-generating mechanisms that are generated in sequential or overlapping stages. For example, in one embodiment the present invention provides an expandable device that can be positioned at a desired target location within a patient to generate mechanical ablation damage. After a predetermined amount of mechanical ablation has occurred, additional ablation damage is generated by a different source, such as energy delivery, drug delivery, or inflammatory material delivery. In this respect, the overall ablation scarring can be better controlled by utilizing the ablation techniques that are most appropriate at specific phases of a technique or locations within a patient.

### RELATED APPLICATIONS

This application claims priority to U.S. Provisional Application Ser. No. 60/617,260 filed Oct. 8, 2004 entitled Implant To Drive Two-Stage Scar Generation In Pulmonary Veins And Left Atrium For Treating Atrial Fibrillation; and U.S. Provisional Application Ser. No. 60/664,925 filed Mar. 24, 2005 entitled Two-Stage Ablation Of Tissue Around Pulmonary Veins To Treat Atrial Fibrillation; the contents of which are hereby incorporated by reference.

### BACKGROUND OF THE INVENTION

This invention is related to implants used to treat atrial fibrillation. Typically, these implants are used to create a scar line through the wall of the ostium of the pulmonary veins or of the atrial wall just inside the atrium from the pulmonary veins. If properly positioned, these scars have the effect of blocking electrical conduction through the tissue of the wall. Blocking this electrical conduction, particularly around the ostia of the pulmonary veins, is known to be effective in stopping either the triggering or maintenance of atrial fibrillation.

Several examples of this type of scar generating implant have been described in previously filed U.S. Patent Publication Nos. 2003-0055491; 2004-0215186 and 2004-0220655, each of which are incorporated by reference herein. As seen in the referenced applications, mechanisms of scar generation include: mechanical pressure necrosis, mechanical cutting, material reaction, and electrical ablation.

While these scar generating techniques are effective, improvements can be made. For example, while RF energy ablation adequately ablates the target tissue, it can also easily char the surface tissue or cause the water in the tissue to boil, causing significant trauma to the ablated tissue. This damage becomes more likely as the depth of the burn increases and can result in more aggressive healing responses at the ablation site. Furthermore, this aggressive healing response can become a clinical problem if it occurs in and causes narrowing of the pulmonary veins.

Scar generation can also be effective by using drugs or any type of material that is toxic or inflammatory to the tissue. These drugs or materials can be generally referred to as scar generating materials. Like the electrical ablation methods, scar generating materials can adequately ablate the tissue to which it is exposed, but have some disadvantages. For example, it can be difficult with scar generating material to create a deep scar within tissue without accommodating for migration of the drug or material into undesired areas (e.g., adjacent structures or the blood stream). In other words, the delivery of the drug or material must be highly controlled and precise so as to avoid introduction of a drug dosage or of a scar generating material that either does not reach its intended location (i.e., is not delivered deep enough into the tissue) or disperses so much as to become essentially ineffective.

The mechanical scar generation techniques which are described in the aforementioned applications are excellent for creating scar lines through the walls of the pulmonary veins around the ostia with no readily apparent stenosis (at least not in animal models). However, variations in the tissue properties of the target implant site, e.g., differences in tissue strength, tissue thickness and tissue elasticity, likely require the options of different types, models, sizes, etc. of mechanical implant devices in order to adequately address all potential variations in tissue properties among likely patients. In this regard, the animal studies performed to evaluate different models of devices that are based on mechanical scar generation have shown the walls of the target implant site to be consistently highly compressed even in the areas where scarring through the wall thickness has not been fully achieved.

For at least these reasons, there is a need for a system that creates the desired electrical block in the cardiac tissue by ablating the necessary tissue while minimizing the risk of ablating too much or too little of the cardiac tissue. There is also a need for a system that minimizes the risk of ablating structures beyond the targeted cardiac wall.

### OBJECTS AND SUMMARY OF THE INVENTION

It is an object of the present invention to overcome the limitations of the prior art.

It is another object of the present invention to provide an ablation device that more precisely creates scars within target tissue.

It is yet another object of the present invention to provide an ablation device that minimizes unwanted tissue damage to a patient.

It is yet another object of the present invention to provide an ablation device that more reliably ablates through a target tissue.

It is yet another object of the present invention to provide an ablation technique that can better compensate for variations within the target tissue.

It is another object of the present invention to reduce the different sizes and configurations of devices necessary for different patients.

One preferred embodiment of the present invention seeks to provide a mechanical implant configured to utilize at least two different scar-generating mechanisms that are generated in sequential or overlapping stages. For example, the present invention provides an expandable device that can be positioned at a desired target location within a patient to generate mechanical ablation damage. After a predetermined amount of mechanical ablation has occurred, additional ablation damage is generated by a different source, such as RF, drug delivery, or material delivery. In this respect, the overall ablation scarring can be better controlled by utilizing the ablation techniques that are most appropriate at specific phases of a technique or locations within a patient.

### BRIEF DESCRIPTION OF THE DRAWINGS

FIG. 1 illustrates a perspective view of a prosthesis according to a preferred embodiment of the present invention;

FIG. 2 illustrates a side view of the prosthesis of FIG. 1 within a pulmonary vein;

FIGS. 3A and 3B illustrate an enlarged view of a portion of the prosthesis of FIG. 2;

FIG. 4 illustrates an enlarged view of a prosthesis according to a preferred embodiment of the present invention;

FIG. 5 illustrates a perspective view of a prosthesis according to another preferred embodiment of the present invention;

FIG. 6 illustrates a side view of the prosthesis of FIG. 5 within a pulmonary vein;

FIG. 7 illustrates a side view of a prosthesis according to another preferred embodiment of the present invention; and

FIG. 8 illustrates a graph of example release profiles according to another preferred embodiment of the present invention.

### DETAILED DESCRIPTION OF THE INVENTION

Generally, the present invention provides a method and apparatus (also referred to as a prosthesis or implant in this specification) to more precisely create an electrical-blocking scar that reduces or eliminates atrial fibrillation. More specifically, the invention improves the precision of the scar creation and reduces the negative side effects of the previously known ablation techniques. It does this by utilizing a combination of multiple ablation techniques. Since different single ablation techniques have different advantages and disadvantages, multiple techniques can be used in sequence or in an overlapping manner to maximize their advantages and minimize their drawbacks. Thus, with the present invention, a more precise scar can be reliably created to block electrical signals from otherwise propagating through target tissue.

For example, in one embodiment, a mechanical force caused by a prosthesis or implant may be initially used to generate scarring through a portion of the thickness of the target tissue, followed by the application of ablative energy (e.g. Radio Frequency) to the prosthesis to cause scarring through the remaining thickness. Since the mechanical force scars at least a portion of the target tissue first, less ablative energy is needed to complete the scar, thereby minimizing unintended damage or charring otherwise caused by the ablative energy.

In another embodiment, the mechanical force can again be initially used, followed by the delivery or release of a material or drug to the target tissue. Again, since the mechanical force scars a portion of the target tissue thickness, less material or drugs are needed, thereby reducing unintended damage to surrounding tissue areas and minimizing risks of complications that may otherwise be present with higher drug concentrations.

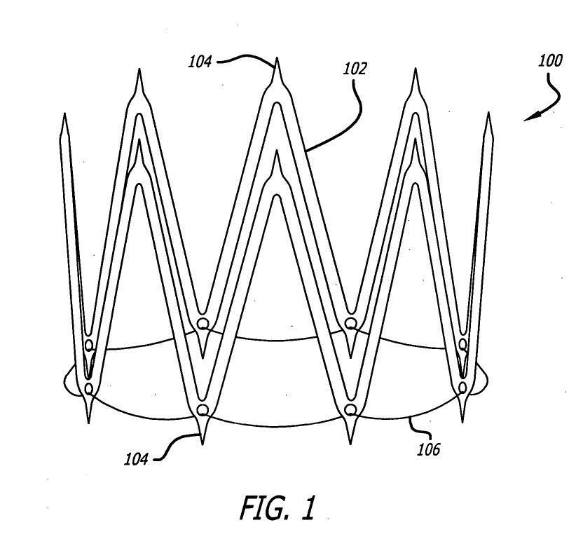

In a more specific example, FIG. 1 illustrates a preferred embodiment of a self-expanding prosthesis 100 according to the present invention. The prosthesis 100 is configured to mechanically generate a scar at least partially through the thickness of a tissue wall. The remaining thickness is then scarred by the application of ablative energy such as RF energy. In this respect, the prosthesis 100 can be described as having a first ablation stage and a second ablation stage. While these ablation stages are preferably performed in a generally sequential order, portions of these ablation stages can also overlap each other.

As seen best in FIG. 1, the prosthesis is composed of a plurality of “zig-zag” struts 102 that are configured to exert a mechanical pressure against the desired target tissue. The peaks where each strut 102 connects to the next includes an anchoring barb 104 which is shaped to pierce the target tissue and therefore provide anchoring support to the prosthesis 100. A wire 106 is fixed to the peaks of struts 102 on one side of the prosthesis, creating a circular region that further exerts a narrow area of pressure on the target tissue.

Preferably, the prosthesis 200 is formed by cutting the shapes of the prosthesis body into a nitinol tube having an internal diameter of about 0.155 inches and an outer diameter of about 0.197 inches. The struts 102 can preferably be cut to have a width of about 0.020 inches and a length of about 0.400 inches, while the wire 106 is preferably cut to a width of about 0.006 inches and a length between struts 102 of about 0.350 inches.

The prosthesis 100 can preferably be cut and polished over a cylindrical rod having a diameter of 26 mm for support. It may be desirable to polish the prosthesis 100 before and after forming (e.g. cutting) to minimize cracking in the forming process. A prosthesis 100 having the previously described example dimensions may be appropriate for a target having a diameter of 20 mm, such as a pulmonary vein.

Preferably, the prosthesis 100 is delivered percutaneously to a target tissue, by constraining the prosthesis 100 within a delivery catheter or small diameter sleeve. Examples of possible delivery systems can be found in U.S. application Ser. No. 10/792,110, the contents of which are incorporated herein by reference.

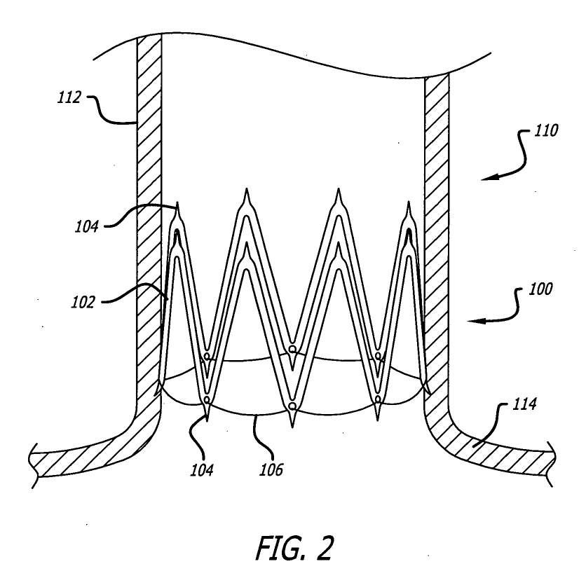

In the first ablation stage, the prosthesis 100 causes mechanical scarring by expanding against the target tissue, such as the pulmonary vein 110, as seen in FIG. 2. The prosthesis 100 continually presses against the wall 112, gradually expanding into, or cutting into, the thickness of the wall 112. As the prosthesis expands into the wall 112 of the pulmonary veins 110, a few millimeters of tissue or neointima forms around the prosthesis 100, effectively encasing the struts 102 within the wall 112.

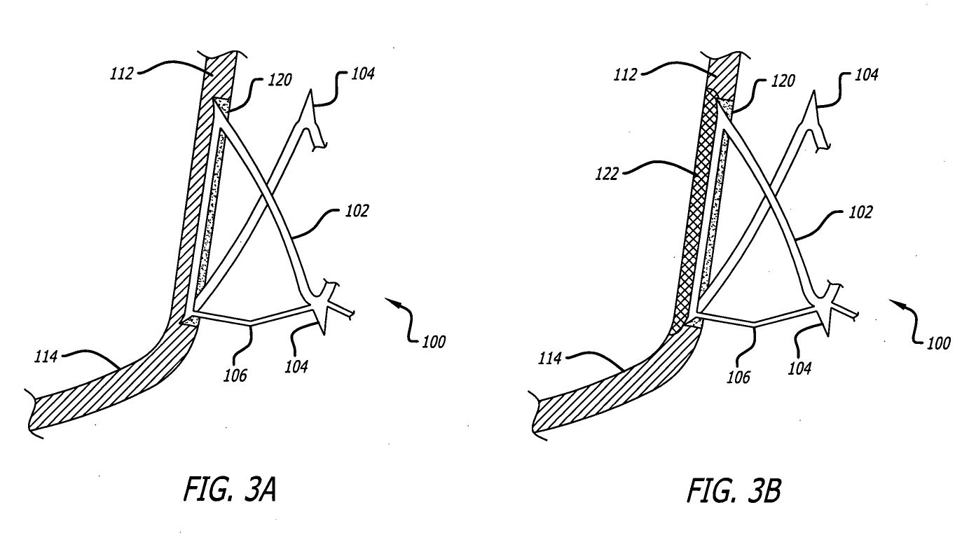

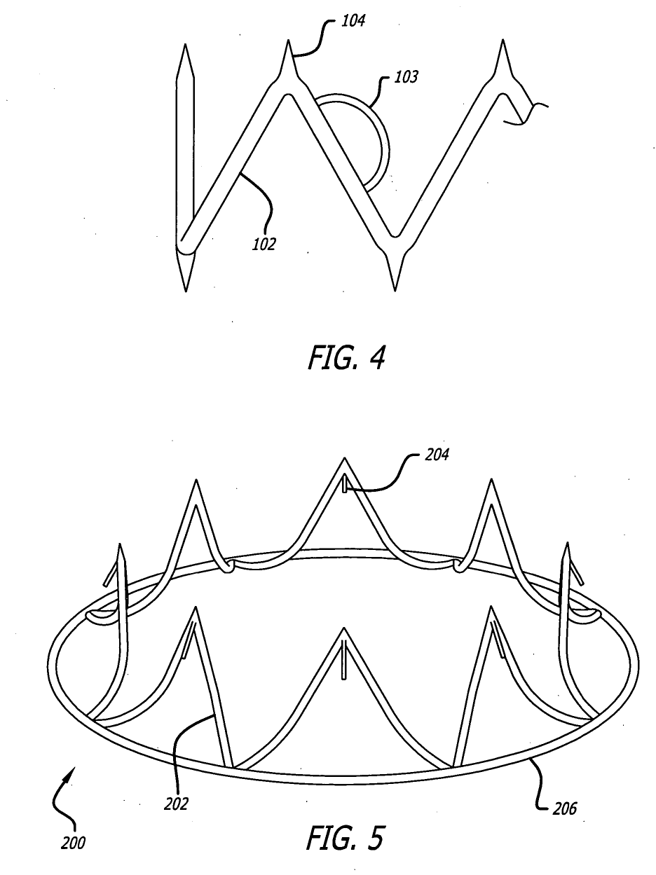

After about a month of this mechanical pressure, the prosthesis 100 will have preferably cut through a large portion of the thickness of the wall 112, creating a mechanically scarred area 120, as seen in FIG. 3A. However, the exact thickness of the scarred area 120 will vary based on a variety of factors, such as the thickness of the wall 112 and the pressure exerted by the prosthesis 100. The remaining unscarred thickness of the wall 112 is likely to be tightly stretched over the prosthesis 100, leaving the remaining wall thickness to be about 1-2 mm.

This remaining thickness of the wall 112 can be ablated during the second ablation stage in which an ablative energy source such as RF is applied to the prosthesis 100, causing tissue damage 122 through the remaining thickness of the wall 112, as seen in FIG. 3B. Since this remaining thickness of the wall 112 is first reduced during the first ablation stage, a relatively smaller amount of ablative energy is required to fully penetrate the wall thickness. For example, a prosthesis can be mostly coated with an insulating coating, having only the wire 106 around the perimeter of the device at the ostium having bare metal in tissue contact. In such an example, the prosthesis diameter may be about 20 mm and the ablative power may be about 40-70 watts of RF power delivered for about two minutes to yield an effective burn around the perimeter of the device.

It should be noted that the advantages of applying a reduced amount of ablative energy can similarly be achieved if the prosthesis 100 simply compresses the target tissue into a thinner configuration, instead of mechanically cutting or pushing into the tissue. In this respect, a thinner amount of tissue is present, reducing the amount of ablative energy needed to create scar tissue completely through the wall 112. In this situation, only one mechanism of ablation may be necessary.

Having a thinner target wall thickness requiring ablation can enable the use of a relatively low ablative energy (e.g. reducing the voltage, current, or application time from values typically used for procedures with energy ablation alone). This can reduce or otherwise eliminate some of the known disadvantages associated with energy ablation. For example, high temperature gradients seen through the thickness of a thicker wall can lead to high tissue impedance, resulting burns on the wall surface, and surrounding tissue damage. These problems can be avoided or greatly reduced when the wall thickness to be ablated is minimized by partial mechanical ablation or compression of the wall. Additionally, a lower ablation energy minimizes the risk of a proliferative response that can lead to stenosis of the pulmonary vein. In this respect, the prosthesis 100 provides a first and second ablation stage to more reliably create an electrical-blocking scar, while minimizing undesirable negative side effects.

As seen in FIG. 4, the prosthesis may include a lead wire 103 having a loop shape that is configured to remain at least partially outside of the target tissue and preferably within the left atrium. Preferably, this lead wire 103 exerts little force on the tissue to minimize it from becoming aggressively embedded. However, an endothelial layer may form over at least part of the wire 103 after the first ablation stage.

To perform the second ablation stage, the lead wire 103 is located angiographically during a second percutaneous procedure and connected to an ablative power supply. Alternatively, the lead wire 103 may be initially positioned through the septum of the heart or atrial wall to facilitate accessing it during the second ablation stage. Such positioning of the lead wire 103 is especially desired when the target is initially accessed trans-septally.

The ablation of the target area by the second ablation stage can be further controlled by coating the struts 102 and barbs 104 with an insulating coating, leaving only the wire 106 electrically exposed to cause ablation. In this respect, a more narrow area of ablation can be generated during the second ablation stage.

In another preferred embodiment according to the present invention, the second ablation stage can be performed by delivering a scar-generating material, such as a drug or chemical, by an ablative coating on at least a portion of the prosthesis 100. Preferably, this ablative coating is applied onto at least a portion of the prosthesis 100, such as the wire 106, followed by a second biodegradable coating. The second biodegradable coating acts to encase the ablative coating and delay its ablative action until the second biodegradable coating has degraded.

In one embodiment, the mechanical ablation generated by the prosthesis 100 during the first ablation stage preferably occurs over about 24 weeks. Hence, it is preferred that the second biodegradable coating delay the delivery of at least a substantial portion of the scar-generating drug of the ablative coating during this time. Such a release delay of the scar-generating drug can allow a scar layer to form behind the prosthesis 100 (i.e. within ablated area 120). This scar tissue can help maintain the integrity of the tissue when the scar-generating drug is released. Additionally, the presence of this scar tissue helps shield the ablative coating from blood flow that may otherwise remove or dilute a portion of the scar-generating drug. Thus, the amount of scar-generating drug within the ablative coating can be further minimized, while the risk of a thrombotic reaction within the blood stream due to the scar-generating drug can be further reduced.

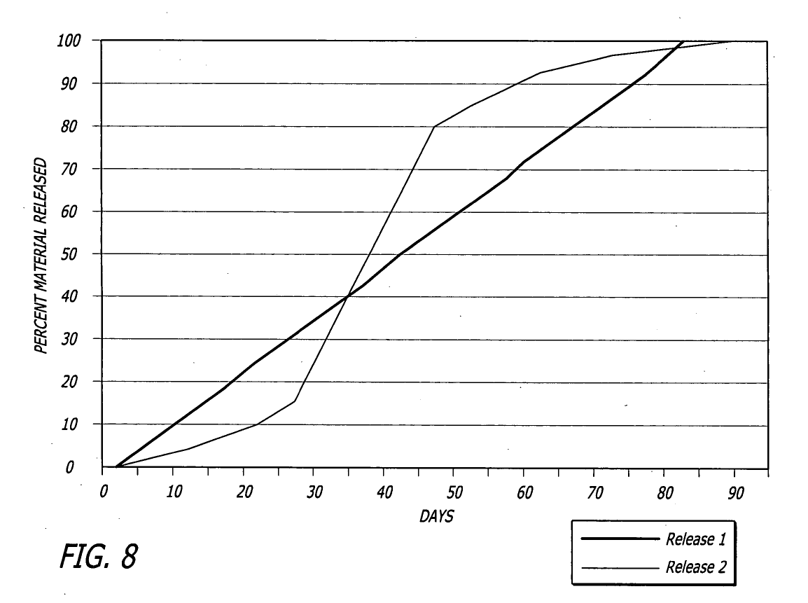

Table 1 below provides 2 sample drug or material release profiles as measured in the number of days after implantation of the prosthesis 100 and by the percentage of material or drug released from the prosthesis 100. This data has also been plotted in the graph shown in FIG. 8 to more clearly illustrate the rates of each release profile.

In the first example release profile, “Release 1” in FIG. 8, the material is released in at a relatively even or constant rate, starting from almost the first day of implant. By comparison, the second example release profile, “Release 2” in FIG. 8, releases the material at a relatively low rate until almost 30 days after implantation of the prosthesis 100, at which point the release rate dramatically increases. In other words, release profile 2 initially releases very little drugs or material into the target tissue. However after about a month, a significantly larger amount of drugs are released into the target tissue.

TABLE 1

| | Release Profile 1 | Release Profile 2 |
|---|---|---|
| Time After Implant | (Percentage Material | (Percentage Material |
| (Days) | Released) | Released) |
| 0 | 0 | 0 |
| 5 | 2 | 6 |
| 10 | 4 | 12 |
| 15 | 7 | 18 |
| 20 | 10 | 25 |
| 25 | 15 | 31 |
| 30 | 31 | 37 |
| 35 | 47 | 43 |
| 40 | 64 | 50 |
| 45 | 80 | 56 |
| 50 | 85 | 62 |
| 55 | 89 | 68 |
| 60 | 93 | 75 |
| 65 | 95 | 81 |
| 70 | 97 | 87 |
| 75 | 98 | 93 |
| 80 | 99 | 100 |
| 85 | 100 | 100 |
| 90 | 100 | 100 |

It should be noted that the advantages of applying a reduced amount of scar-generating drug can similarly be achieved if the prosthesis 100 simply compresses the target tissue into a thinner configuration, instead of mechanically cutting or pushing into the tissue. In this respect, a thinner amount of tissue is present, requiring less scar-generating drug to achieve a concentration so as to create scar tissue completely through the wall 112. In this situation, only one mechanism of ablation may be necessary.

Preferably, the biodegradable coating prevents the ablative coating from being released or otherwise acting on the target tissue until the prosthesis 100 has pushed into the wall 112 of the pulmonary vein 110. Thus, exposure of the scar-generating material of the ablative coating to the blood is minimized. Some biodegradable coating materials include Polydioxanone, Poliglecaprone, Polyglactin, Polyorthoester, or some of the other biodegradable materials mentioned elsewhere in this specification.

The ablative coating may include biodegradable polymers that cause an inflammation and ultimately scarring. Examples of such polymers include 100% poly l-lactide, 100% poly d,i-lactide, 85% poly d, l-lactide/15% caprolactone. These examples are produced by Alkermes in their Medisorb line of bio-absorbable polymers.

Similarly, the biodegradable polymer ablative coating may include a relatively less inflammatory, higher molecular weight biodegradable material over a lower molecular weight, more inflammatory, layer which breaks down faster. Thus, the higher molecular weight layer can shield the lower molecular weight layer, allowing a smaller inflammatory and therefore ablative response to be initially implemented, while a larger response can begin later.

The ablative coating may also be an ablative drug carried in a polymer substrate. Such ablative drugs include alkylating agents such as Cis-Platin, Cyclophosphamide, Carmustine, Fluorouracil, vinblastine and Methotrexate. These ablative drugs also include antibiotics such as tetracycline, actinomycin, polidocanol, Doxorubicin, D-Actinimycin and Mitomycin. Another possible type of ablative drugs are surfactants such as Sotradecol or Polydocanol.

Further, combinations of drugs or materials may be used to ablate tissue. For example, one drug may be included to act on collagen or elastin, while another drug may be included to act on muscle tissue.

The amount and depth of scarring caused by ablation can be adjusted by increasing or decreasing the amount of ablative drugs or material in an ablative coating. This scarring depth can especially be adjusted in regards to the amount of scarring caused by other ablation techniques used in the procedure. For example, if for a specific design of the device, a first ablation stage mechanically ablates about half of a target tissue thickness in a typical manner, the ablative drugs can be reduced in that design to an appropriate level to ablate the remaining thickness.

The ablative coating may further include materials such as glutaraldehyde, metallic copper, and copper compounds held in a polymer matrix. Materials such as glutaraldehyde and copper compounds within a matrix can be eluted from a non-biodegradable polymer matrix or delivered in a biodegradable polymer matrix. Metallic copper, on the other hand, may be provided in wire form around the perimeter of the implant so as to be shielded from blood flow contact by a biodegradable coating until the prosthesis 100 becomes fully embedded within the target tissue (e.g. the wall 112).

These ablative, scar-generating drugs can be loaded into a biodegradable polymer substrate to form the ablative coating. For example, such polymers include Polyesteramide produced by Medivas or Gliadel (polyanhydride,poly[1,3-bis(carboxyphenoxy)propane-co-seacic-acid](PCPP-SA)matrix) produced by Guilford pharmaceutical. In this example, the Polyesteramide and the Gliadel can release the scar-generating drugs progressively as they are absorbed by the target tissue.

Non-biodegradable polymers can also be used for the ablative coating, such as Biospan segmented polyurethane produced by Polymertech. In this example, the Biospan releases the scar-generating drug/material by diffusion after the second biodegradable over coating has degraded.

Additional drug delivery methods known in the art are also possible. For example, the scar-generating drugs can be encapsulated into degradable spheres that are released from the prosthesis 100.

Returning to an embodiment that utilizes mechanical ablation, it is noted that mechanical ablation can often be hindered by the tissue composition of the target area. For example, the proximal region of the pulmonary vein 110 is typically comprised of a venous tissue layer on the inside of the pulmonary vein 110, followed by a surrounding muscular tissue layer. The venous tissue (comprised largely of elastin and collagen) is thinner, significantly tougher and less elastic than the outer muscular tissue.

Thus, mechanical ablation mechanisms, such as the prosthesis 100, may need to produce a relatively high expansive force in order to push into the tissue layers of the pulmonary vein 110. Such mechanical ablation can be facilitated by utilizing a different ablative mechanism during a first ablation stage to damage or ablate the tough venous tissue layer.

For example, a first ablation stage may include applying ablative energy (e.g. RF) to the prosthesis 100 after delivery at a target location. Preferably, only enough ablative energy is provided to ablate through the venous tissue layer, allowing the mechanical expansive force of the prosthesis 100 during the second ablation stage to press into and through the relatively softer muscle tissue layers. Again, since relatively low levels of ablative energy can be used, the risk of causing a proliferative response which can lead to stenosis is also low.

In another example, the first ablation stage may include applying an ablative drug or material in a coating, as previously discussed in this specification. Preferably, the drug or material can be selected to quickly break down the venous tissue layer. For example, a collagenase material like Tripcyn or Papain can be used as a coating on the prosthesis 100 to break down the collagen in the venous tissue layer, allowing the prosthesis 100 to easily expand into the muscular tissue layer and complete the desired scar. Similarly, an elastase material such as the active enzymes found in dental bacteria such as strepmutans could be effective in breaking down the elastin layer.

While the previous examples have been described in terms of first ablation stages and second ablation stages, it should be understood that some ablative techniques may overlap or may even begin or end at the same time. For example, when an ablative drug is used for a first ablation stage and an expansive mechanical ablative technique is used for a second ablation stage, both ablation techniques will likely begin to operate at about the same time. However, the ablative drug will mostly cease damaging tissue before the mechanical ablation. In this respect, non-overlapping, sequential ablation techniques are not necessarily required and in some preferred embodiments, the use of different overlapping ablation techniques is preferred. Additionally, more than two ablation techniques may be used in a single technique. For example, 3 or even 4 ablation techniques may be used.

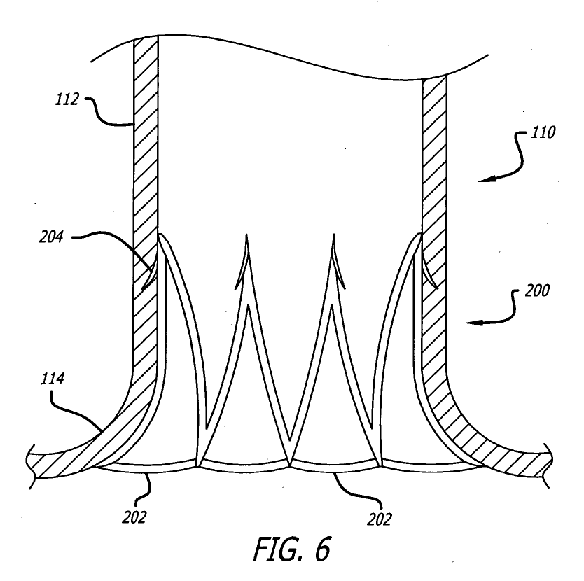

FIG. 5 illustrates another preferred embodiment of a prosthesis 200 according to the present invention. The prosthesis 200 is generally similar to the previously described prosthesis 100, including a plurality of struts 202 aligned to form “zig-zag” peaks and valleys, anchoring barbs 204 disposed on the peaks of one side of the prosthesis 200, and a wire 206 connecting the struts 202 on the other side of the prosthesis 200. However, the struts 202 curve or flare outwardly towards the wire 206, preferably forming an expanded shape that matches the ostium 114 of the pulmonary vein 110, as seen in FIG. 6. In this respect, one portion of the prosthesis 200 is positioned to contact a proximal portion of the pulmonary vein 110 while another portion is positioned to contact the ostium 114 or atrial wall outside of the pulmonary vein.

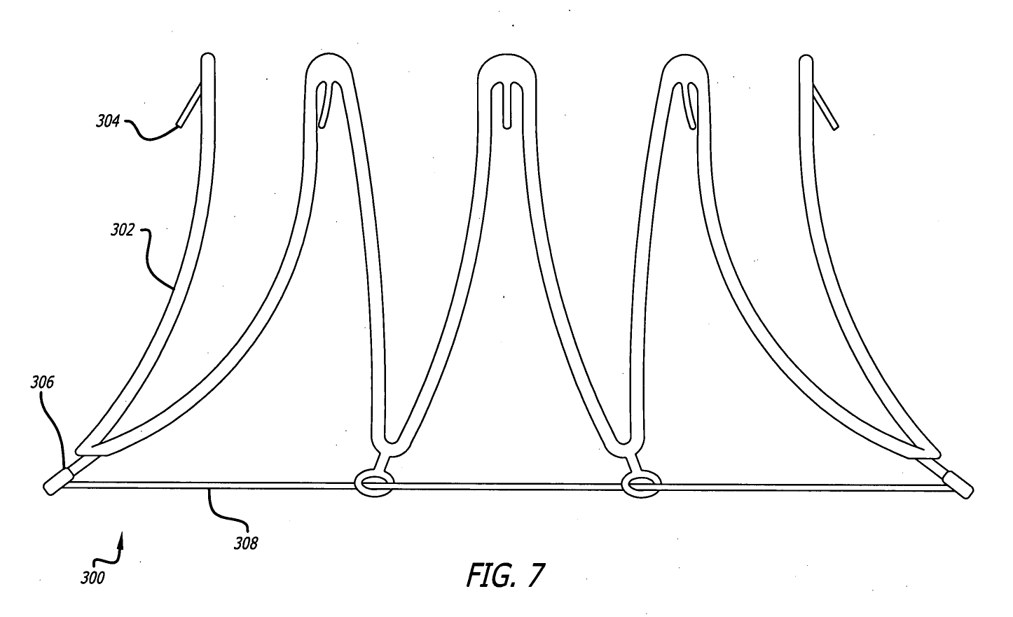

In an alternative preferred embodiment seen in FIG. 7, a wire 308 from the prostheses 300, can be a distinct, separate component, as opposed to being of an integral construction. In such a configuration, the wire 308 can be retained with eyelets 306 on the ends of the struts 302 (the end opposite of the anchoring barbs 304), allowing the wire 308 to be composed of a variety of different materials. One possible preferred embodiment includes the wire 106 composed of copper and over coated with a biodegradable coating to prevent exposure of the copper to the bloodstream until the wire has become embedded in the wall. This can help minimize the risk of clot formation on the copper wire.

For example, the wire 308 may be composed of a biodegradable polymer which includes an ablative material, such as those previously discussed in this application. In this respect, the volume of the polymer is not constrained by the maximum thickness that can be coated onto a metal wire. Instead, the primary volume constraint is the volume of the cross section of the wire 308 itself. Therefore a greater amount of polymer can be included, allowing a greater loading of ablative material and possibly a greater delay in releasing the ablative material.

In another example, the wire 308 can be composed of cobalt palladium or a nickel palladium alloy. The ferro-magnetic properties of these example metals and alloys allow for inductively heating the wire 308 to cause ablation. Preferably, this inductive heating can be performed during a second ablation stage, after a mechanical first ablation stage. Since the prosthesis 308 is preferably embedded within the target tissue when the inductive heating is caused, clot formation within the blood flow of the pulmonary vein 110 is minimized.

Additionally, the example metals and alloys tend to self regulate their temperature when exposed to the appropriate magnetic fields, as described in U.S. Patent Application No. 2002/0183829, the contents of which are herein incorporated by reference. This temperature regulation can help ensure that only a desired amount of heat is used to generate ablation, minimizing unwanted damage and complications.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method of creating a conduction block in tissue comprising:
placing an implant in a target location in a patient, said target location having a wall thickness;
inducing a first scar through at least a portion of said wall thickness using a first scarring mechanism;
inducing a second scar through a remaining portion of said wall thickness using a second scarring mechanism; and
wherein said first scarring mechanism is different than said second scarring mechanism.

2. The method of claim 1, wherein said inducing a first scar through at least a portion of said wall thickness using a first scarring mechanism includes mechanically ablating a target tissue with said implant.

3. The method of claim 2, wherein said inducing a second scar through a remaining portion of said wall thickness using a second scarring mechanism includes delivering a drug.

4. The method of claim 3, wherein said drug is an aklylating agent.

5. The method of claim 3, wherein said drug is an antibiotic.

6. The method of claim 3, wherein said drug is a biodegradable polymer.

7. The method of claim 3, wherein said delivering a drug includes degrading a biodegradable coating prior to releasing said drug.

8. The method of claim 2, wherein said inducing a second scar through a remaining portion of said wall thickness using a second scarring mechanism includes delivering an ablative energy to said implant.

9. The method of claim 8, wherein said delivering an ablative energy to said implant includes supplying ablative energy to a lead wire of said implant.

10. The method of claim 8, wherein said delivering an ablative energy to said implant includes supplying ablative radio frequency energy to a lead wire of said implant.

11. The method of claim 1, wherein said inducing a first scar through at least a portion of said wall thickness using a first scarring mechanism is performed during a first time and inducing a second scar through a remaining portion of said wall thickness using a second scarring mechanism is performed during a second time.

12. The method of claim 11, wherein said first time and said second time are sequential.

13. The method of claim 11, wherein said first time and said second time overlap.

14. A prosthesis for generating a scar within a patient comprising:
a prosthesis body having an expanded state and a compressed state;
a first ablation component disposed on said prosthesis so as to induce tissue ablation during a first period of time; and
a second ablation component disposed on said prosthesis so as to induce tissue ablation during a second period of time.

15. The prosthesis of claim 14, wherein the first ablation component is a mechanically ablative component.

16. The prosthesis of claim 14, wherein the second ablation component is a tissue inflaming substance ablative component.

17. The prosthesis of claim 14, wherein the second ablation component element includes an ablative energy supply.

18. The prosthesis of claim 14, wherein said second period of time is consecutive with said first period of time.

19. The prosthesis of claim 14, wherein said second period of time overlaps at least a portion of said first period of time.

20. The prosthesis of claim 14, wherein said prosthesis body includes a plurality of struts connected to a circular wire and positioned to contact an adjacent strut.

21. A method of creating a conduction block in tissue comprising:
placing an implant in a target location in a patient, said target location having a tissue thickness;
damaging at least a portion of said tissue thickness using a first ablating mechanism;
damaging a remaining portion of said tissue thickness using a second ablating mechanism; and
wherein said first ablating mechanism is different than said second ablating mechanism.

22. The method of claim 21, wherein said damaging at least a portion of said tissue thickness using a first ablating mechanism occurs during a first time period and wherein said damaging a remaining portion of said tissue thickness using a second ablating mechanism occurs during a second time period.

23. The method of claim 22, wherein said first time period and said second time period are sequential.

24. The method of claim 22, wherein said first time period and said second time period overlap.

25. The method of claim 21, wherein said damaging at least a portion of said tissue thickness using a first ablating mechanism includes applying a mechanical pressure against said tissue thickness.

26. The method of claim 21, wherein said damaging at least a portion of said tissue thickness using a first ablating mechanism includes delivering a drug to said tissue thickness.

27. The method of claim 21, wherein said damaging at least a portion of said tissue thickness using a first ablating mechanism includes applying an ablative energy to said implant.

28. The method of claim 21, wherein said damaging a remaining portion of said tissue thickness using a second ablating mechanism includes applying a mechanical pressure against said tissue thickness.

29. The method of claim 21, wherein said damaging a remaining portion of said tissue thickness using a second ablating mechanism includes delivering a drug to said tissue thickness.

30. The method of claim 21, wherein said damaging a remaining portion of said tissue thickness using a second ablating mechanism includes applying an ablative energy to said implant.

31. The method of claim 21, wherein said placing an implant in a target location in a patient includes positioning said implant at least partially within a pulmonary vein.

32. A method of creating a conduction block in tissue comprising:
placing an implant in a target location in a patient, said target location having a wall thickness;
reducing said wall thickness with a first tissue disruption mechanism of said implant;
damaging a remaining thickness of said target location with a second tissue disruption mechanism; and
wherein a tissue disruption capability of said second tissue disruption mechanism is inversely related to a tissue disruption capability of said first disruption mechanism.

33. A method according to claim 32, wherein said first tissue disruption mechanism is a mechanical disruption mechanism.

34. A method according to claim 33, wherein said second tissue disruption mechanism includes an ablative drug.

35. A method according to claim 34, wherein a greater reduction in said wall thickness achieved by said mechanical disruption mechanism reduces the amount of ablative drug required in said second tissue disruption mechanism.

36. A method according to claim 34, wherein said damaging of said remaining thickness is achieved through delayed release of said ablative drug.

37. A method according to claim 3, wherein the delivering of a drug includes a delayed release of said drug.

38. A prosthesis according to claim 16, wherein said tissue inflaming substance ablative component is a delayed release drug.

39. A method according to claim 26, wherein said delivering a drug to said tissue thickness includes a delivering said drug through delayed delivery.

40. A method of creating scar lines through the wall of tissue of a pulmonary vein comprising:
providing a prosthesis having an expanded state and a compressed state;
pressing a portion of said prosthesis into tissue around an ostium of said pulmonary vein when said prosthesis is in its expanded state;
allowing a neointimal layer to substantially cover said portion of said prosthesis; and
releasing a substantial portion of an ablative material disposed in said portion of said prosthesis only after a formation of said neointimal layer.

41. A method according to claim 40, wherein an initial portion of ablative material is released prior to the releasing of a substantial portion of said ablative material.

42. A method according to claim 40, wherein said ablative material is a scar generating medical substance.

43. A device for creating scar lines through a tissue wall of a pulmonary vein comprising:
a support structure having an expanded state and a compressed state;
a tissue engagement structure disposed on said support structure;
said tissue engagement structure being loaded with an ablative material; and
said tissue engagement structure having a barrier structure preventing release of a substantial portion of said ablative material until after a neointimal layer is formed on said tissue engagement structure.

44. A device according to claim 43, wherein said barrier allows release of an initial portion of said ablative material prior to formation of said neointimal layer, said initial portion being less than said substantial portion.

45. A device according to claim 43, wherein said ablative material is a scar generating medical substance.
